(19) United States
(12) Patent Application Publication
(10) Pub. No.: US 2018/0125947 A1
(43) Pub. Date: May 10, 2018

(54) TREATMENT OF RETINAL DETACHMENT

(71) Applicants: INSERM (INSTITUT NATIONAL DE LA SANTÉ ET DE LA RECHERCHE MÉDICALE), Paris (FR); UNIVERSITÉ PARIS DESCARTES, Paris (FR); UNIVERSITÉ PIERRE ET MARIE CURIE (PARIS 6), Paris (FR); UNIVERSITÉ PARIS DIDEROT - PARIS 7, Paris (FR); ASSISTANCE PUBLIQUE-HÔPITAUX DE PARIS (APHP), Paris (FR)

(72) Inventors: Emilie PICARD, Paris (FR); Yves COURTOIS, Paris (FR); Francine BEHAR-COHEN, Paris (FR)

(21) Appl. No.: 15/574,600

(22) PCT Filed: May 19, 2016
(86) PCT No.: PCT/EP2016/061281

§ 371 Date: Nov. 16, 2017

(30) Foreign Application Priority Data
May 20, 2015 (EP) 15305761.7

(51) Int. Cl. A61K 38/40; A61P 27/02

(52) U.S. Cl. CPC A61K 38/40; A61P 27/02

(57) ABSTRACT

The present invention relates to the Transferrin (Tf) or an active fragment thereof for use in the treatment of retinal detachment and/or for use in the treatment of retinal detachment related to other diseases.

### FIELD OF THE INVENTION

The present invention relates to the Transferrin (Tf) or an active fragment thereof for use in the treatment of retinal detachment and/or for use in the treatment of retinal detachment related to other diseases.

### BACKGROUND OF THE INVENTION

Accumulation of fluid into the potential space between the neurosensory retina and the underlying retinal pigment epithelium in the presence of a retinal detachment (RD), break or tear represent the characteristic feature of the disease [Mitry D et al., 2010]. Nevertheless, its composition and its involvement in the physiopathology events remain still little known. The mainstay of treatment is early intervention; restoration of retinal anatomy and blood supply are crucial in this disorder. Functional remodelling of the retina is essential to achieve an acceptable visual recovery [Sakai T et al., 2013]. To date, various molecular and cellular mechanisms have been suggested regarding the detachment related retinal ischemia and apoptosis of photoreceptors [Murakami Y et al., 2013]. The main therapeutic challenge in RD is to limit photoreceptor cells damage.

### SUMMARY OF THE INVENTION

In this study, the inventors explored iron and Tf content in vitreous and sub-retinal fluids collected from patients undergoing retinal surgery and compared to samples from non-retinal detachment cases and correlated to pre- and post-operative examinations.

Thus, the invention relates to the Transferrin (Tf) or an active fragment thereof for use in the treatment of retinal detachment and/or for use in the treatment of retinal detachment related to other diseases.

### DETAILED DESCRIPTION OF THE INVENTION

### Protein and Uses Thereof

A first object of the invention relates to the Transferrin (Tf) or an active fragment thereof for use in the treatment of retinal detachment (RD) and/or for use in the treatment of retinal detachment related to other diseases.

The inventors saw that in case of retinal detachment, many photoreceptors die. Thus, the Transferrin (Tf) or an active fragment thereof can be used to prevent photoreceptors death during retinal detachment.

Thus, the invention also relates to the Transferrin (Tf) or an active fragment thereof to prevent photoreceptors death during retinal detachment.

According to the invention, retinal detachment can be related to diseases that included but are not limited to systemic diseases, ocular diseases, retinal vascular diseases, choroidal vascular diseases (see for example Roy F H, Lippincott Williams; Wilkins 2002 and www.cybersight.org).

According to the invention, systemic diseases include but are not limited to aspergillosis, blood diseases, lymphoma, polyarteritis, renal diseases, syphilis, Marfan's syndrome.

According to the invention, ocular diseases include but are not limited to retinal necrosis, choroidal or retinal tumors, myopia, exudative vitreoretinopathy, Schwartz syndrome, diabetic retinopathy, retinopathy of prematury, severe uveitis and age-related macular degeneration.

According to the invention, retinal and/or choroidal vascular diseases include but are not limited to retinal necrosis, choroidal or retinal tumors, exudative vitreoretinopathy, diabetic retinopathy, retinopathy of prematury, Eales diseases and exudative age-related macular degeneration.

According to the invention the Transferrin (Tf) or an active fragment thereof may be used in case of RD associated with drug side effects.

According to the invention the Transferrin (Tf) or an active fragment thereof may be used in case of trauma to prevent photoreceptors death during RD.

According to the invention the Transferrin (Tf) or an active fragment thereof may be used in case of RD secondary to cataract surgery.

According to the invention the Transferrin (Tf) or an active fragment thereof may be used in case of RD secondary to previous RD.

According to the invention the Transferrin (Tf) or an active fragment thereof may be used in case of retinal detachment-autosomal dominant or x-linked.

In a particular embodiment, the retinal detachment is a rhegmatogenous retinal detachment, exudative, serous, tractional retinal detachment, or secondary retinal detachment. Rhegmatogenous retinal detachment (RRD) is characterized by the separation of the vitreous gel from the retina without any damage that leads to retinal tear formation due to the presence of strong vitreoretinal adhesions. Tractional RD which is induced by proliferative vitreoretinopathy (PVR), contractile (vitreo-, epi-, intra-, or sub-) retinal membranes that pull the neuroretina away from the RPE by mechanical effect without causing retinal tear. Tractional RD may occur in a number of pathologic conditions, such as proliferative diabetic retinopathy, sickling hemoglobinopathies and retinopathy of prematurity at an advanced stage. The exudative/serous RD occurs when the subretinal fluid accumulates in the absence of retinal tear. Any pathology that affects choroidal vascular permeability or that damages the RPE can lead to fluid accumulation in the subretinal space. The three types of RD are not mutually exclusive. For example, vitreo-retinopathy exhibits both RRD and tractional RD. The scope of this study is mainly limited to RRD, also called “primary/simple RD”. The term “complex RD” is used to describe the RD complicated by PVR.

Thus, the Transferrin (Tf) or an active fragment thereof can be used to prevent photoreceptors death during “primary/simple RD”, as well as “complex RD”.

As used herein, the term “retinal detachment” denotes a disorder of the eye in which the retina peels away from its underlying layer of support tissue. Initial detachment may be localized or broad, but without rapid treatment the entire retina may detach, leading to vision loss and blindness. The degree of functional recovery has been associated with age, degree of myopia (axial length of the globe), but also with type of RD, duration/persistence and composition of SRF. It is almost always classified as a medical emergency. Permanent damage may occur if the detachment is not repaired within 24-72 hours. Since photoreceptor cell death starts within the first twelve hours and peaks at 2-3 days following RD, it is important to perform surgery as quickly as possible, especially when the macula is detached (Cook B, Lewis G P, Fisher S K, et al. Invest Ophthalmol Vis Sci. 1995; 36(6):990-996. Hisatomi T, Sakamoto T, Murata T, et al. Am. J. Pathol. 2001; 158(4):1271-8; Arroyo J G, Yang L, Bula D, et al. Am J Ophthalmol. 2005; 139(4):605-610.). If the SRF persists under the macula for more than 4-6 days, RRD is associated with significantly lesser chance of visual recovery (Khanzada M A, Wahab S, Hargun L D. Pak J Med Sci. 2014; 30(3):525-529; van Bussel E M, van der Valk R, Bijlsma W R, et al. Retina. 2014; 34(10):1917-1925.) In eyes that present complete retinal reattachment but in which the visual acuity recovery is incomplete or delayed, the quality of the fluid seems to be important.

Cone photoreceptor apoptotic cell death occurs very early after detachment and could be stopped by reattachment of the retina (Sakai T, Calderone J B, Lewis G P, et al. Invest Ophthalmol Vis Sci. 2003; 44(1):416-425; Fisher S K, Stone J, Rex T S, et al. Prog Brain Res. 2001; 131:679-698.).

The only treatment for RRD is surgery aiming at closing all the breaks with minimal damage. Drainage or absorption of the subretinal fluid (SRF) allows anatomical retinal reattachment to regain/maintain visual function. This is accomplished either by bringing the eye wall closer to the detached retina (scleral buckling) or by pushing the detached retina toward the eye wall (vitrectomy and intraocular tamponade with a gas bubble or silicon oil). Sealing of the breaks is accomplished by creating a strong chorioretinal adhesion around the breaks using LASER photocoagulation or cryocoagulation. Currently, no role exists for medical care in the treatment of tractional RD. Depending on its extent, surgical interventions: scleral buckling techniques and/or with vitrectomy, can be offered to patients in order to relieve vitreoretinal traction. These RD are mainly managed with specific etiologic treatment in case of tumors (chemotherapy, proton beam therapy . . . ), or with medical treatments: anti-inflammatory agents in inflammatory conditions (Andreoli C M, Foster C S. Int Ophthalmol Clin. Spring 2006; 46(2):111-122), antibiotics for infectious etiologies, or anti-VEGF in Coats disease (Kase S, Rao N A, Yoshikawa H, et al. Invest Ophthalmol Vis Sci. 2013; 54(1):57-62; 4. Gaillard M C, Mataftsi A, Balmer A, et al. Retina 2014; 34(11):2275-81.). New treatments are also emerging, as the management of chronic central serous chorioretinopathy with mineralocorticoid receptor antagonist (Bousquet E, Beydoun T, Zhao M, et al. Retina. 2013; 33(10):2096-102; Dirani A, Matet A, Beydoun T, et al. Clin Ophthalmol 2014; 8:999-1002).

Thus, in a particular embodiment and according to the invention, the Transferrin (Tf) or an active fragment thereof is administrated to a patient in need thereof in emergency that is to say in the 24 hours after the retinal detachment or in the 12 hours after the retinal detachment. Thus, the invention relates to the Transferrin (Tf) or an active fragment thereof to complete (be an adjunct to) surgical intervention.

Iron is a potential cell death enhancer due to the fact that excess of the labile ferrous form is a high generator of oxidative stress via Fenton reaction. Intraocular foreign iron body (siderosis) and hemorrhages provokes retinal degeneration (Karpe G. Doc Ophthalmol. 1948; 2(1):277-296.). While RPE cells have high iron sequestration capacity (Hadziahmetovic M, Song Y, Wolkow N, et al. Am J Pathol. 2011; 179(1):335-348), photoreceptors are extremely sensitive to iron labile overload (Rogers B S, Symons R C, Komeima K, et al. Invest Ophthalmol Vis Sci. 2007; 48(1):438-45.).

As used herein, the term “Transferrin” or “Tf” denotes a blood plasma protein for iron delivery. Transferrin is a glycoprotein that binds iron very tightly but reversibly. Although iron bound to transferrin is less than 0.1% (4 mg) of the total body iron, it is the most important iron pool, with the highest rate of turnover (25 mg/24 h). Transferrin has a molecular weight of around 80 kiloDaltons and contains 2 specific high-affinity Fe(III) binding sites. The affinity of transferrin for Fe(III) is extremely high (10.23 M−1 at pH 7.4) but decreases progressively with decreasing pH below neutrality.

In a particular embodiment and according to the invention, human Transferin (hTf) or an active fragment thereof may be used. A sequence for human Transferrin gene is deposited in the database NCBI under accession number NM 001063.

In one embodiment, retinal detachment can occur in neurodegenerative diseases such as AMD. Thus, in this case, Transferrin can be used to prevent the retinal detachment.

Thus, the invention also relates to Transferrin (Tf) or an active fragment thereof for use in the prevention of retinal detachment in patient suffering from retinal disorders.

In another embodiment, the invention relates to the Transferrin (Tf) or an active fragment thereof for use in the treatment of retinal detachment and/for use in the treatment of retinal detachment related to other diseases.

As used herein, the term “an active fragment” denotes a fragment of a protein that retains the activity of the complete protein or has been modified to have increased binding property as compared to the full length native “Transferrin”. For example, an active fragment of the Transferrin denotes a fragment of the protein which conserves the capacity to binding the iron.

As used herein, the terms “treating” or “treatment”, denotes reversing, alleviating, inhibiting the progress of, or preventing the disorder or condition to which such term applies, or one or more symptoms of such a disorder or condition.

According to the invention, the term “patient” or “individual” to be treated is intended for a human or non-human mammal (such as a rodent (mouse, rat), a feline, a canine, or a primate) affected or likely to be affected with retinal detachment or with retinal detachment related to other diseases. Preferably, the patient is a human.

The capacity to binding the iron of the Tf or the active fragment thereof will become evident to the skilled person by implementing a simple test to evaluate the binding of iron of said proteins. For instance iron is readily removed from transferrin by incubation in a buffer containing ImMNTA, 1 mM EDTA, 0.5 M sodium acetate pH 4.9 The apoprotein is concentrated to a minimum volume on a centricon 10 (amicon), then diluted and reconcentrated twice with water and twice with 0.1 NKCl. The apoprotein has a tendency to precipitate in pure water but redissolves in 0.1 NMKCL.

In a preferred embodiment, said active fragment of Tf comprises at least 75% identity over said Tf, even more preferably at least 80%, at least 85%, at least 90%, at least 91%, at least 92%, at least 93%, at least 94%, at least 95%, at least 96%, at least 97%, at least 98%, at least 99%.

Tf or active fragment thereof may be produced by any technique known per se in the art, such as, without limitation, any chemical, biological, genetic or enzymatic technique, either alone or in combination(s).

Knowing the amino acid sequence of the desired sequence, one skilled in the art can readily produce a relevant part of the Tf or the active fragment thereof, by standard techniques for production of proteins. For instance, they can be synthesized using well-known solid phase method, preferably using a commercially available protein synthesis apparatus (such as that made by Applied Biosystems, Foster City, Calif.) and following the manufacturer's instructions.

Alternatively, Tf or active fragment thereof can be synthesized by recombinant DNA techniques as is now well-known in the art. For example, these fragments can be obtained as DNA expression products after incorporation of DNA sequences encoding the desired polypeptide into expression vectors and introduction of such vectors into suitable eukaryotic or prokaryotic hosts that will express the desired protein or fragment of the protein, from which they can be later using well-known techniques.

Tf or active fragment thereof can be used in a vector, such as a membrane or lipid vesicle (e.g. a liposome).

In specific embodiments, it is contemplated that the Tf or the active fragment thereof used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 45 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

### Nucleic Acids, Vectors, Recombinant Host Cells and Uses Thereof

A second aspect of the invention relates to a nucleic acid molecule encoding the Transferrin or an active fragment thereof for use in the treatment of retinal detachment and/or for use in the treatment of retinal detachment related to other diseases.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

These nucleic acid molecules may be obtained by conventional methods well known to those skilled in the art, in particular by site-directed mutagenesis of the gene encoding the native protein. Typically, said nucleic acid is a DNA or RNA molecule, which may be included in a suitable vector, such as a plasmid, cosmid, episome, artificial chromosome, phage or viral vector.

So, a further object of the present invention relates to a vector and an expression cassette in which a nucleic acid molecule of the invention is associated with suitable elements for controlling transcription (in particular promoter, enhancer and, optionally, terminator) and, optionally translation, and also the recombinant vectors into which a nucleic acid molecule in accordance with the invention is inserted. These recombinant vectors may, for example, be cloning vectors, or expression vectors.

The terms “vector”, “cloning vector” and “expression vector” mean the vehicle by which a DNA or RNA sequence (e.g. a foreign gene) may be introduced into a host cell, so as to transform the host and promote expression (e.g. transcription and translation) of the introduced sequence.

Any expression vector for animal cell may be used, as long as a gene encoding a polypeptide or chimeric derivative of the invention can be inserted and expressed. Examples of suitable vectors include pAGE107, pAGE103, pHSG274, pKCR, pSG1 beta d2-4) and the like.

Other examples of plasmids include replicating plasmids comprising an origin of replication, or integrative plasmids, such as for instance pUC, pcDNA, pBR, and the like.

Other examples of viral vector include adenoviral, retroviral, herpes virus and AAV vectors. Such recombinant viruses may be produced by techniques known in the art, such as by transfecting packaging cells or by transient transfection with helper plasmids or 30 viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. No. 5,882,877, U.S. Pat. No. 6,013,516, U.S. Pat. No. 4,861,719, U.S. Pat. No. 5,278,056 and WO 94/19478.

Examples of promoters and enhancers used in the expression vector for animal cell include early promoter and enhancer of SV40, LTR promoter and enhancer of Moloney mouse leukemia virus, promoter and enhancer of immunoglobulin H chain and the like, and promoter specific of RPE or Müller cells or photoreceptors, microglial cells.

The invention also includes gene delivery systems comprising a nucleic acid molecule of the invention, which can be used in gene therapy in vivo or ex vivo. This includes for instance viral transfer vectors such as those derived from retrovirus, adenovirus, adeno associated virus, lentivirus, which are conventionally used in gene therapy. This also includes gene delivery systems comprising a nucleic acid molecule of the invention and a non-viral gene delivery vehicle. Examples of non viral gene delivery vehicles include liposomes and polymers such as polyethylenimines, cyclodextrins, histidine/lysine (HK) polymers, etc.

Another object of the invention is also a prokaryotic or eukaryotic host cell genetically transformed with at least one nucleic acid molecule according to the invention.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

Preferably, for expressing and producing the proteins, and in particular the Transferrin, eukaryotic cells, in particular mammalian cells, and more particularly human cells, will be chosen.

Typically, cell lines such as CHO, BHK-21, COS-7, C127, PER.C6 or HEK293 could be used, for their ability to process to the right post-translational modifications of the derivatives.

The construction of expression vectors in accordance with the invention, the transformation of the host cells can be carried out using conventional molecular biology techniques. The V-ATPase c-subunit derivatives of the invention, can, for example, be obtained by culturing genetically transformed cells in accordance with the invention and recovering the derivative expressed by said cell, from the culture. They may then, if necessary, be purified by conventional procedures, known in themselves to those skilled in the art, for example by fractionated precipitation, in particular ammonium sulphate precipitation, electrophoresis, gel filtration, affinity chromatography, etc.

In particular, conventional methods for preparing and purifying recombinant proteins may be used for producing the proteins in accordance with the invention.

### Pharmaceutical Compositions

A third object of this invention is a pharmaceutical composition which includes a therapeutically effective amount of at least the Transferrin or an active fragment thereof according to the invention, along with at least one pharmaceutically acceptable excipient for use in the treatment of retinal detachment and/or for use in the treatment of retinal detachment related to other diseases.

Alternatively, the pharmaceutical composition of the invention may contain a therapeutically effective amount of a nucleic acid according to the invention or a plasmid or a vector that contains at least one nucleic acid sequence that codes for the Transferrin of the invention, along with at least one adjuvant and/or a pharmaceutically acceptable excipient.

Said vector may be used in gene therapy.

By a “therapeutically effective amount” is meant a sufficient amount of the chimeric derivative of the invention to treat retinal detachment and/or for use in the treatment of retinal detachment related to other diseases at a reasonable benefit/risk ratio applicable to any medical treatment.

It will be understood that the total daily dosage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The active products of the invention (proteins or vectors) may be administered for the treatment of retinal detachment related to other diseases.

The therapeutically effective amount of the active product of the invention [proteins or vectors (constructions)] that should be administered, as well as the dosage for the treatment of a pathological condition with the proteins and/or pharmaceutical compositions of the invention, will depend on numerous factors, including the age and condition of the patient, the severity of the disturbance or disorder, the method and frequency of administration and the particular peptide to be used.

The presentation of the pharmaceutical compositions that contain the proteins or vectors (constructions) of the invention may be in any form that is suitable for administration, e.g., solid, liquid or semi-solid, such as creams, ointments, gels or solutions, and these compositions may be administered by any suitable means, for example, orally, parenterally, inhalation or topically, so they will include the pharmaceutically acceptable excipients necessary to make up the desired form of administration. A review of the different pharmaceutical forms for administering medicines and of the excipients necessary for obtaining same may be found, for example, in the “Tratado de Farmacia Gal nica” (Treatise on Galenic Pharmacy), C. Faul i Trillo, 1993, Luz n 5, S. A. Ediciones, Madrid.

In the pharmaceutical compositions of the present invention for oral, sublingual, subcutaneous, intramuscular, intravenous, transdermal, local, pulmonary, eye drop, intraocular (intravitreous, sub-retinal) or rectal administration, the active principle, alone or in combination with another active principle, can be administered in a unit administration form, as a mixture with conventional pharmaceutical supports, to animals and human beings. Suitable unit administration forms comprise oral route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms, intraocular and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The Tf or the active fragment thereof according to the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

For intraocular administration, the composition according to the invention may be by electroporated for example through a device described in the patent application WO2006123248, WO03030989.

The Tf or the active fragment thereof of the invention may be formulated as a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

An additional object of this invention relates to the Tf or the active fragment thereof or of vectors that contain at least one sequence that codes for Tf or the active fragment thereof for use in the treatment of retinal detachment and/or for use in the treatment of retinal detachment related to other diseases.

In addition, the invention provides a method for treating retinal detachment and/or for treating retinal detachment related to other diseases which consists of administering to a patient a therapeutically effective amount of Tf or an active fragment thereof, or of a vector containing at least one DNA sequence that codes for Tf or the active fragment thereof.

The invention will be further illustrated by the following figures and examples. However, these examples and figures should not be interpreted in any way as limiting the scope of the present invention.

### FIGURES

FIG. 1: Iron metabolism is modified during retinal detachment.

A-C. Determination of iron concentration (A), transferrin (Tf) quantity (B) and Tf saturation (C) in vitreous of patient controls and with retinal detachment (RD). Iron content was determined by ICP-MS and Tf concentration was reported to protein concentration. Mann-Whitney test (n=10-12); A: ** p=0.0041; B: * p=0.0271; C: ** p=0.0062

D: Evaluation of incidence of duration of RD on iron concentration in subretinal fluids (SRF) and vitreous from RD patients. The duration of RD was separated into 2 groups with duration inferior and superior of 7 days. Mann-Whitney test (n=3 per time).

E: Visual acuity recovery in function of iron concentration (left graph) and Tf saturation (right panel) in subretinal fluids (SRF) and vitreous from RD patients. Visual acuity recovery was reported to delta log MAR (post-operative minus pre-operative) and separated in 2 groups relative to the mean of delta log MAR. Mann-Whitney test (n=3 per delta log MAR).

FIG. 2: Transferrin protects rhodopsin proteins of rats retinas exposed to iron.

Rat retinas are exposed to iron during 2 days, collected 2 days after and proteins are extracted. Rods pigment (Rhodopsin) are quantified by Western Blot (Mann Whitney test).

### EXAMPLES

### Example 1

Material & Methods

Human Sample Collections

The present prospective case-control study was approved by the medical committee of Hospital Jules Gonin (Lausanne, Swizterland) and Hospital Hôtel-Dieu (Paris, France). For vitreous and sub-retinal fluids, after detailed explanation of the purpose and methods of the study, written and informed consent was obtained from the subjects before participating. Fluids samples were collected during surgery using a convenient sampling approach. Post-mortem eyes were obtained in ana-pathological services of Hospital Jules Gonin The study was conducted in adherence to the tenets of the Declaration of Helsinki.

Patients and Design

Of a total of 25 patients for fluids analysis, 13 patients with RD fulfilled the inclusion criteria (age 64 years; SD: 10.8; 5 females, 10 males), and 12 patients control (age 71.2 years; SD: 9.5, 8 females, 5 males) were matched to these patients. All patients were thoroughly examined by slit lamp inspection. Patients with signs of central serous chorioretinopathy, glaucoma, infection or intra-ocular hemorrhage were excluded. Patients with no signs of RD but presented vitreoretinal traction syndrome (1), macular hole (5) or epiretinal membrane (6) were considered as controls. Undiluted vitreous samples were collected at the start of the three-port vitrectomy. Before the infusion line was opened, the vitreous sample was collected in a syringe connected to the vitreotome and then immediately frozen at −20° C. until biochemical analysis.

For 6 patients with RD (age 61.5; SD: 4,3; 3 females, 3 males) macula OFF, sub-retinal fluids were also collected at same time than vitreous. SRF fluid drainage was performed using a 26-gauge needle attached to a 2-ml syringe without the plunger. The needle was inserted perpendicular to the sclera, and the upsurge of fluid was seen in the transparent hub of the needle. For these patients, duration of RD defined as the time between the onset of symptoms and surgery was noted. Visual acuities were recorded before and after surgery and converted to logarithm of the minimum angle of resolution (log MAR). Visual recovery was calculated as post-operative log MAR minus pre-operative log MAR. The mean of delta log MAR was used to divided SRF in 2 groups.

For all samples, hemoglobin contamination was verified with hemoglobin colorimetric assay, and only one sample with hemoglobin concentration upper than limit of detection was discarded.

Iron Analysis

VH and LSR samples (50 μL) were diluted with 1.95 mL of HNO3 1% solution containing 1 ng/mL Indium as internal standard. Samples are analyzed using an inductively coupled plasma mass spectrometer (ICP M S, 7700 Series, Agilent, Santa Clara, Calif., USA). Quantitative analysis of iron (Fe) was carried out by external calibration using 7 standards with concentration ranging from 10 to 1,000 ng/mL. The method is assessed using internal and external quality controls, analysis of initial calibration, verification standard, procedural blanks, and duplicate samples.

Transferrin and proteins were quantified with ELISA kit (AssayPro, St-Charles, USA) and Micro BCA assay kit (Pierce) respectively. Saturation of transferrin was calculated as total iron content (μmol/L)/TIBC (μmol/L). TIBC measures the amount of iron that can potentially be stored in Tf, and correspond to the capacity of 1 mol of Tf (80 000 Da) to bind 2 mol of ferric iron and was calculated (μmol/L) as Tf concentration (g/l)×10̂6 X (2/80 000).

Tissues Processing

After enucleation, a small incision was made in the pars plana and each eye was fixed in a combination of 4% PAF and 0.5% glutaraldehyde in 0.1M phosphate buffer for several days. A block of tissue ranging from 1 to 1.5 cm long and 0.5 cm wide containing the RD was processed as serial 5 μm thick paraffin sections. Sections were stained with Perls Prussian blue for iron with or not DAB staining amplification as described.

Results

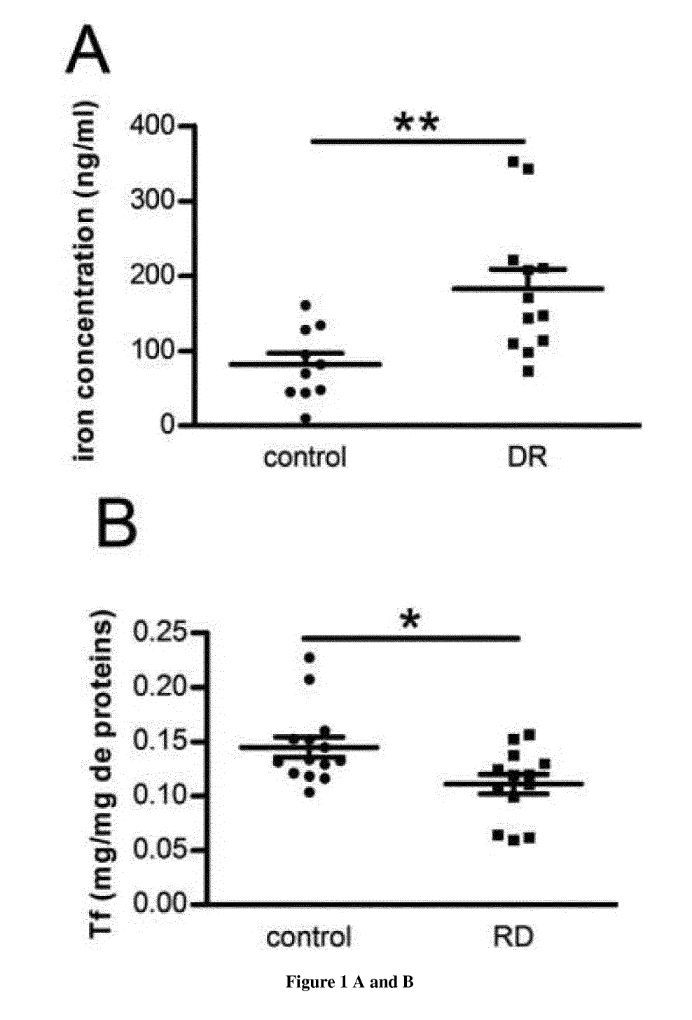

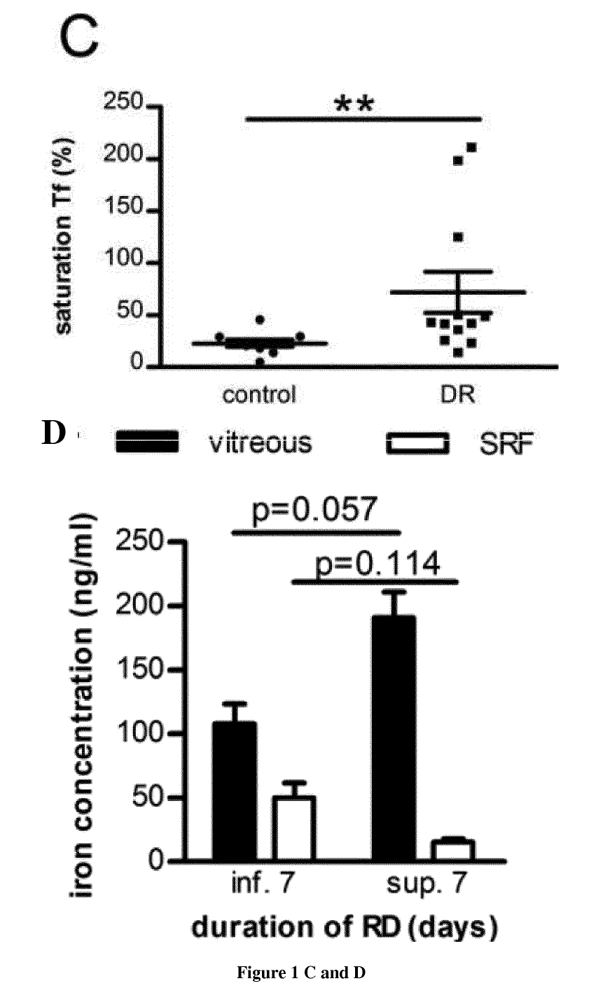

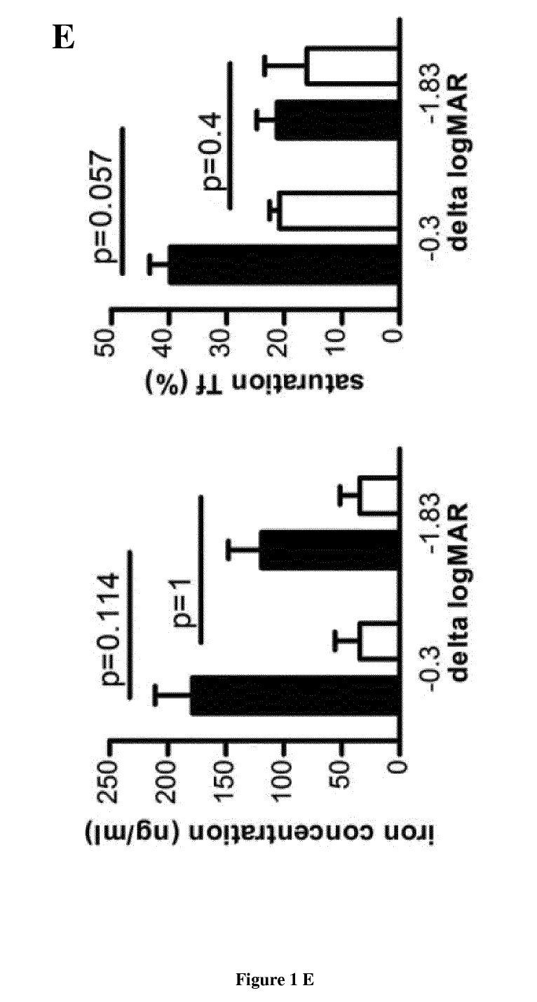

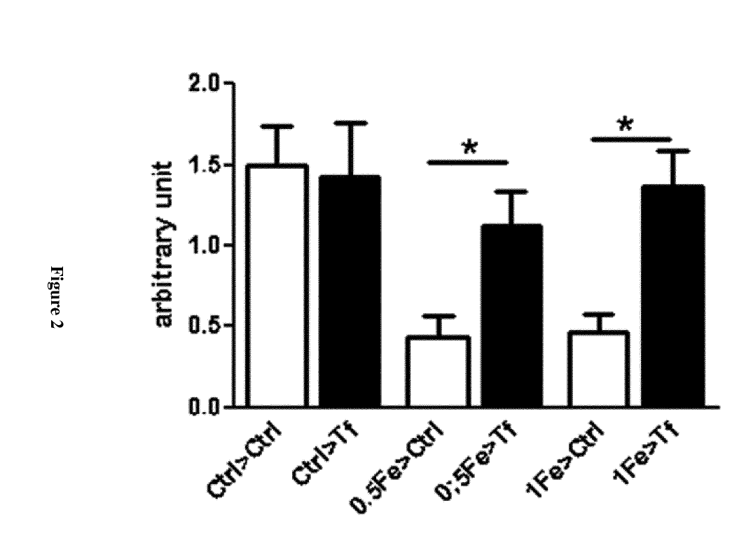

Iron concentration in RD vitreous was significantly higher compared to control vitreous (FIG. 1A). Tf concentration which was reported to protein concentration was significantly decreased in RD vitreous compared to control vitreous (FIG. 1B). In FIG. 1C, Tf saturation in iron was consequently significantly higher on RD vitreous than control vitreous. Iron staining (data not shown) on retina detached demonstrated iron deposits in remaining PRs layers (arrow). Iron deposit was also observed in retinal pigmented epithelium (arrowhead) and in cells accumulated in retinal space (data not shown). Subretinal fluids (SRF) and vitreous from same RD patients were also analyzed for iron metabolism. In RD SRF, there was 38.5 ng/ml±10.38 and was significantly lower than vitreous (RD vitreous: 135.7 ng/ml±2.71; Wilcoxon test, p=0.0313, n=6). The iron metabolism was reported to duration of RD and visual acuities recorvery in these samples. When the duration of RD was superior at 7 days, iron concentration was higher in vitreous and lower in SRF (FIG. 1D). A higher visual acuities recovery corresponding to a lower delta log MAR (FIG. 1E) was correlated to a half not significant decrease in vitreous (black bar) iron concentration (left graph) and saturation of Tf (right graph), whereas in SRF (white bar), there were not different.

Discussion

Cell death occurs very early after retinal detachment and the quality and presence duration of the fluid seems to be important [Sakai T et al., 2003 and Fisher S K et al., 2001]. Resulting from RPE barrier breakdown, different toxic compounds present in the serum may also account for the induction of photoreceptor death [Hassel B et al., 1994 and Tomkins O et al., 2007] Here, we found iron in SRF with presence of phagocytic cells loaded with iron in retinal detached area, hypothesized implication of iron accumulation in photoreceptors death during RD. Iron is highly toxic for photoreceptors, especially cones [Rogers B S, Symons R C, Komeima K, et al. Invest Ophthalmol Vis Sci. 2007; 48(1):438-45]. In vitreous of RD patients, iron concentration was higher and correlated with duration of RD and inversely with visual acuities recovery. Currently, surgery is performed to restore visual acuity but not always with successful [Ross W H, Visual recovery after macula-off retinal detachment. Eye 2002, 16, 440-446]. The loss of cells continues even after reattachment and was dependent on the speed for intervention. Additional neuroprotective treatments could improve the final visual outcomes of patients with RD. Rhegmatogenous RD (RRD) incidence is 13.7/100 000/year and patients with RRD on the first eye have a 100 times greater risks of developing RRD on the second eye [Hajari J N et al., 2014]. In vitreous of RD, Tf content was significantly decreased and iron saturation percentage was significantly increased, supposing a decrease in iron sequestering capacity and thus its antioxidant effect. Notwithstanding, the percentage of Tf iron saturation decreased with the best visual recovery. Tf treatment during RD surgery could be a good strategy to decrease iron accumulation and therefore cells death.

### Example 2

Material & Methods

Retinal Explants

Adult rats Wistar were sacrificed and eyes enuclated were rinsed in sterile PBS and placed in DMEM High Glucose (10% fetal calf serum (FCS), 1% penicillin-streptomycin, 1% L-glutamine, and 0.1% fungizone). Rat retinas were then cut into four wedges whereas mice retinas were used entire which was transferred to a membrane (0.22 μm) with the ganglion cells facing the membrane. The membrane was placed into six wells culture plate and 3 ml of medium was added on the bottom. On the top of the retina, 30 μl of medium was added supplemented or not with iron (FeSO₄) during 2 days. Then, retina were rinsed with medium and 30 μl of medium with or not human apotransferrin (50 mg/ml) was added.

Historesin

Retinas were fixed with 4% PAF, 0.5% glutar-aldehyde in PBS for 20 minutes. After fixation, samples were washed, dehydrated and transferred into the infiltration solution of the Leica Historesin embedding kit overnight at 4° C. Samples were embedded in resin and 5 mm thick sections passing in the medium of the explants were collected and stained with 1% Toluidin Blue solution. Sections were then observed on a Leitz microscope and photographed with a Leica camera.

Immunohistochemistries

Retinas were fixed for 20 minutes switch 4% paraformaldehyde in PBS, washed with PBS, infiltrated in gradients sucrose series and then, mounted in Tissue Tek O.C.T. Immunohistochemistry was performed on 10 mm thick sections passing in the medium of the explants. Cryosections were incubated with different primary antibodies: rabbit polyclonal specific for the Light subunit of Ferritin (P. Santambrogio); rabbit anti-GFAP (Dako); rabbit; mouse anti-CD68 (Bio-Rad AbDSerotec GmbH); goat anti-4-Hydroxynonenal (4-HNE) Rods and cones were respectively labeled with anti-rhodopsin (Rho4D2) and anti-arrestin (Abcam) The corresponding Alexa-conjugated secondary antibodies were used to reveal the primary antibodies, and sections were counterstained with 4.6-diamidino-2-phenylindole (DAPI). The sections were viewed with a fluorescence microscope (BX51, Olympus) and photo-graphed using identical exposure parameters for all samples to be compared.

Western Blot

Fresh retina were rinsed and lysed with M-PER buffer. 15 μg of proteins were then electrophoresed through a 4% stacking-12.5% running gel, and transferred onto a nitrocellulose membrane. Membranes were incubated with primary antibodies against rhodopsin (1:5000), anti-actin (1:2000) over night. Antibodies were visualized with horseradish peroxidase conjugated secondary goat anti-rabbit or a rabbit anti-mouse antibody (1:5000-1:10,000). Detection was performed and quantification with ImageJ software.

Results

Retinal organoculture consists to recreate retinal detachment (RD) leading to PR loss as observed in human. Retina is isolated from eye ball and placed in culture serum-free medium. This organotypic ex vivo preparation has advantages over other experimental in vitro systems because it maintains mature neurons in situ and in contact with their normal cellular environment, thereby facilitating physiological interactions. This system permits direct retinal manipulation, allow greater control of retinal environment, provides greater efficiency and timeliness compared with animal models. Indeed, immediately after retinal isolation, PR begin to die, and in low serum medium, retinal explants keeps PR up to 10 days of culture (Fernandez-Bueno I, Fernandez-Sanchez L, Gayoso M J, Garcia-Gutierrez M T, Pastor J C, Cuenca N. Time course modifications in organotypic culture of human neuroretina. Exp Eye Res 2012; 104:26-38).

Rat retinal explants exposed to iron have PRs death increased (FIG. 1). The addition of human apoTf (50 mg/ml) in culture medium preserves retinal histology (FIG. 2A), revealed by immunostaining of both photoreceptors (data not shown). Western Blot of rhodopsin proteins in retinal explants shows a higher level in explants treated with Tf (FIG. 2B). Moreover, inflammation, gliose and oxidative stress increased in iron excess condition are reduced with Tf treatment (data not shown).

Retina from WT mice and mice expressing human transferrin (Tg) are isolated and keep in culture for several days in condition of iron overload. Retinas from Tg mice synthesize Tf that protects cones and rods from death (data not shown).

Organoculture of retina in condition of iron overload mimics retinal detachment, showing photoreceptors cell death, iron accumulation, inflammation, gliosis and iron accumulation. Human apoTf efficiency protects retinal detachment deleterious consequences.

### Example 3

Material & Methods

Animals

Tg mice carrying the long hTf gene (80 kb) comprising its long 5′- and 3′-regulatory sequences were generated from the 803 line previously described [Picard E et al Mol Vis 2008] backcrossed in the C57Bl/6J background. They were screened for the presence of hTf in the blood, using ELISA. All Tg mice used in this study were homozygous for the hTf gene. Control animals were WT C57Bl/6J mice (Janvier). All mice were fed a standard laboratory diet and tap water ad libitum and maintained in a temperature controlled room at 21-23° C. with a 12 h:12 h light-dark photoperiod. Animals were sacrificed by carbon dioxide inhalation. All experimental procedures were submitted and approved by the local ethics committee European Council Charles Darwin, University Paris Descartes. Experiments were performed in accordance with the Association for Research in Vision and Ophthalmology (ARVO) statement for the use of animals in Ophthalmic and Vision Research.

Retinal Detachment Procedure

Mice were anesthetized, pupils were dilated with a topically applied mixture of phenylephrine and mydriaticum. Subretinal injections were realized with a 31 gauge needle connected to a syringe filled with 5 mg/mL sodium hyaluronate. In each experimental eye, approximately one half of the retina was detached.

Historesin

Oriented eyes were fixed with 4% PAF, 0.5% glutaraldehyde in PBS for 2 hours. After fixation, samples were washed, dehydrated and transferred into the infiltration solution of the Leica Historesin embedding kit overnight at 4° C. Samples were embedded in resin and 5 mm thick sections passing trough retinal detachment were collected and stained with 1% Toluidin Blue solution. Sections were then observed on a Leitz microscope and photographed with a Leica camera.

Immunohistochemistries

Retinas were fixed for 2 hours with 4% paraformaldehyde in PBS, washed with PBS, infiltrated in gradients sucrose series and then, mounted in Tissue Tek O.C.T. Immunohistochemistry was performed on 10 mm thick sections pa passing trough retinal detachment. Rods and cones were respectively labeled with anti-rhodopsin (Rho4D2) and anti-arrestin (Abcam) The corresponding Alexa-conjugated secondary antibodies were used to reveal the primary antibodies, and sections were counterstained with 4.6-diamidino-2-phenylindole (DAPI). The sections were viewed with a fluorescence microscope (BX51, Olympus) and photo-graphed using identical exposure parameters for all samples to be compared.

Results

A rat model of RD has already been used to test novel neuro-protective drugs using mechanical retinal detachment and subretinal injection of sodium hyaluronate (Woo T T, Li S Y, Lai W W, Wong D, Lo A C. Neuroprotective effects of lutein in a rat model of retinal detachment. Graefes Arch Clin Exp Ophthalmol 2013; 251:41-51; Xie Z, Chen F, Wu X, et al. Safety and efficacy of intravitreal injection of recombinant erythropoietin for protection of photoreceptor cells in a rat model of retinal detachment. Eye (Lond) 2012; 26:144-152). We implement this technique to assess the therapeutic effect of Tf in RD. The induction of this model combined a mechanical retinal detachment and a subretinal injection of sodium hyaluronate to mimic RD (Hisatomi T, Sakamoto T, Murata T, et al. Relocalization of apoptosis-inducing factor in photoreceptor apoptosis induced by retinal detachment in vivo. Am J Pathol 2001; 158:1271-1278; Matsumoto H, Miller J W, Vavvas D G. Retinal detachment model in rodents by subretinal injection of sodium hyaluronate. J Vis Exp 2013). Photoreceptor degeneration induced in the RD model has the advantage of a reasonable time course from days to weeks.

WT mice and Tg mice expressing human Tf are subretinally injected with sodium hyaluronate and eyes are collected 7 days after for histology and immunostaining. Nuclear layer of photoreceptors (ONL) and retinal pigmented epithelium layer are highly modified in WT mice after retinal detachment compared to control WT mice. Histology of TG mice retina is less degenerated than WT retina (FIGS. 1 and 2). After RD, both photoreceptor cell types are more preserved in Tg mice compared to WT mice (data not shown).

Animal model of retinal detachment reveals retinal degeneration and loss of photoreceptors. However, in situ expression of human Tf in retina protects from retinal detachment effects.

### REFERENCES

Throughout this application, various references describe the state of the art to which this invention pertains. The disclosures of these references are hereby incorporated by reference into the present disclosure.
Fisher S K, Stone J, Rex T S, et al. Prog Brain Res. 2001; 131:679-698.
Hajari J N, Bjerrum S S, Christensen U, et al. Retina. 2014; 34(8):1658-1665.
Hassel B, Iversen E G, Fonnum F. Neurosci Lett. 1994; 167(1-2):29-32.
Mitry D, Fleck B W, Wright A F, Campbell H, Charteris D G. Pathogenesis of rhegmatogenous retinal detachment: predisposing anatomy and cell biology. Retina. 2010; 30:1561-72.
Murakami Y, Notomi S, Hisatomi T, Nakazawa T, Ishibashi T, Miller J W, Vavvas D G. Photoreceptor cell death and rescue in retinal detachment and degenerations. (Murakami et al, 2013); 37:114-40.
Sakai T, Calderone J B, Lewis G P, et al. Invest Ophthalmol Vis Sci. 2003; 44(1):416-425.
Sakai T, Tsuneoka H, Lewis G P, Fisher S K. Remodelling of retinal on- and off-bipolar cells following experimental retinal detachment. Clin Experiment Ophthalmol. 2013; 12246.
Rogers B S, Symons R C, Komeima K, et al. Invest Ophthalmol Vis Sci. 2007; 48(1):438-45
Ross W H, Visual recovery after macula-off retinal detachment. Eye 2002, 16, 440-446
Tomkins O, Friedman O, Ivens S, et al. Neurobiol Dis. 2007; 25(2):367-377.

1-3. (canceled)

4. A method for treating retinal detachment and/or for treating retinal detachment related to other diseases in a patient in need thereof comprising administering to the patient a therapeutically effective amount of Transferrin (Tf) or an active fragment thereof.

5. The method of claim 4, wherein the transferrin is human transferrin.

6. The method of claim 4, wherein the related diseases are selected from the group consisting of systemic diseases, ocular diseases, retinal vascular diseases and choroidal vascular diseases.
